SEQUENCE LISTING (4 sequences)

SEQ ID NO: 1 - DNA, 16 bp, synthetic construct
  source 1..16: mol_type = other DNA; note = ITR forward
cggcctcagtgagcga

SEQ ID NO: 2 - DNA, 21 bp, synthetic construct
  source 1..21: mol_type = other DNA; note = ITR reverse
ggaacccctagtgatggagtt

SEQ ID NO: 3 - DNA, 25 bp, synthetic construct
  source 1..25: mol_type = other DNA; note = Albumin forward
tgaaacatacgttcccaaagagttt

SEQ ID NO: 4 - DNA, 26 bp, Synthetic construct
  source 1..26: mol_type = other DNA; note = Albumin reverse
ctctccttctcagaaagtgtgcatat

(19) United States
(12) Patent Application Publication
(10) Pub. No.: US 2026/0103730 A1
(43) Pub. Date: Apr. 16, 2026

(54) METHOD FOR RELEASING VIRAL VECTORS

(71) Applicant: PCI Biotech AS, Oslo (NO)

(72) Inventors: Morten LUHR, Oslo (NO); Anders HØGSET, Oslo (NO); Karin NORD, Vøyenenga (NO)

(73) Assignee: PCI Biotech AS, Oslo (NO)

(21) Appl. No.: 19/115,362

(22) PCT Filed: Sep. 27, 2023
(86) PCT No.: PCT/EP2023/076807

§ 371 Date: Mar. 26, 2025

(30) Foreign Application Priority Data
Sep. 27, 2022 (GB) 2214112.1
Mar. 17, 2023 (GB) 2303918.3
Jun. 28, 2023 (GB) 2309780.1

(51) Int. Cl. C12N 15/864; C12N 5/07; C12N 5/071; C12N 5/079; C12N 15/861

(52) U.S. Cl. CPC C12N 15/8645; C12N 5/0601; C12N 5/0621; C12N 5/0686; C12N 15/861; C12N 2509/10; C12N 2510/04; C12N 2529/10; C12N 2710/10043; C12N 2710/10052; C12N 2750/14143; C12N 2750/14152

(57) ABSTRACT

The present invention provides a method of releasing viral vectors from cells producing those viral vectors by contacting the cells with a photosensitising agent which is then irradiated to disrupt the plasma membrane of the cells to release the viral vectors which may be collected and/or purified. The product of such methods as well as kits and apparatuses for performing the methods are also provided.

The present invention provides methods of releasing viral vectors, such as adenovirus or adeno-associated virus, from cells which produce them by using photosensitising agents which when activated disrupt the plasma membrane allowing release of the viral vectors. Kits and apparatuses for this purpose are also provided.

Viral vectors (VVs) are of great importance for the introduction of transgenes into cells, for example in gene therapy applications. Mammalian cells in culture are commonly used to manufacture VVs, and are considered production cells for their manufacture. By way of example, the cell line HEK293 is frequently used for this purpose.

In order to produce VVs, production cells are transfected with one or multiple plasmids encoding the genes necessary for production of the VVs (and in most cases the transgene of interest). Following transfection, production cells assemble functional viral vectors that tend to accumulate intracellularly. To selectively purify VVs, the cells must be opened up. Current processes use cell lysis to achieve this.

Purification of VVs from production cells represents a major hurdle to manufacturers, having substantial economic consequences for the industry. Several purification processes have been developed and are in use, but all of these suffer from severe shortcomings such as lack of efficiency and scalability, as well as practical complexity and time-consumption (Ehrke-Schulz et al., 2016, J. Vis. Exp., 107, e52894; Grieger et al., 2016, Molec. Ther., 24 (2), p 287-297). Furthermore, lysis methods generally result in contamination with DNA from the lysed cell. There is therefore a clear need for better procedures to obtain and purify VVs.

The present inventors have developed methods that allow release of VVs from production cells. The method uses photochemical treatment as a means to disrupt the plasma membrane of cells in culture, in particular VV production cells, thereby allowing entrapped VVs to be released into solution for subsequent purification. This photochemical treatment is also referred to as photochemical lysis (PCL) herein. In these methods photosensitising agents are used to generate light-induced chemical reactions (mainly mediated by reactive oxygen species (ROS)) that disrupt the membranes of VV production cells.

Preferably photosensitising agents are selected that anchor in the plasma membrane. Furthermore, to avoid harm to the VVs the photosensitising agents and VVs are preferably selected so that they do not bind to one another.

Illumination is easy to use on the production cells after culture of those cells and one may even use white light, such as artificial light or natural sunlight. This leads to a convenient and scaleable method as the photosensitiser stock solutions can be provided at high concentrations that do not dilute the cell-viral vector solution and irradiation can be performed in a broad range of cell-viral vector volumes and vessels or containers. The method may be coupled with other lysis techniques if necessary. The VVs may be subsequently purified.

Purification of VVs is currently a multistep process that may include chemical lysis of the plasma membrane, hypotonic lysis of the plasma membrane, centrifugation, homogenisation, sonication, freeze-thawing, enzyme digestion (e.g. DNase), and/or liquid chromatography. Photochemical treatment as described herein may be performed jointly with one or multiple of these steps. In particular, the photosensitising agent may be used to aid plasma membrane disruption e.g. by addition to detergent-based lysis buffers, hypotonic solutions, or in the sonication buffer. A detergent as referred to herein may also be referred to as a surfactant.

As shown in the Examples, the photochemical lysis methods of the invention have been shown to develop pores in the treated cells which allow the uptake of small molecules (Example 1, Hoechst 33258 stain) and the release of larger molecules (Example 3, lactate dehydrogenase). Surprisingly, DNA that was present in the cell before illumination was retained in the cell after illumination and did not leak from the cells (Examples 5, 7, 8 and 9). This illustrates the selective permeabilisation achieved by methods of the invention. This provides significant advantages for release of VVs and their collection relative to prior art methods in which contamination with cellular DNA is common, e.g. when detergent lysis is used.

The photochemical lysis method of the invention is illustrated in FIG. 8 which shows the production of pores (as evidenced by the results in the Examples) from which viral vectors may be released without leakage of cellular DNA. Example 6 shows the release of VVs from production cells using methods of the invention.

Thus in a first aspect, the present invention provides a method of releasing a viral vector from a cell in which said viral vector has been produced, comprising:
a) contacting a cell in which said viral vector is present with a photosensitising agent,
b) irradiating said cell with light of a wavelength effective to activate said photosensitising agent, wherein said irradiation is conducted at a dose of light and for a time sufficient to disrupt the plasma membrane of said cell, thereby releasing said viral vector, and
c) optionally collecting and/or purifying said released viral vector.

### Viral Vector

The “viral vector” is a virus that may be used as a vector for delivery of genetic material. Such a viral vector is not simply the nucleic acid material, but corresponds to the final viral form which may include relevant capsid, core and cement proteins. Generally, such a viral vector has been modified for therapeutic purposes. Such modification may be used to modify the naturally occurring virus to add desired properties and minimise possible pathogenicity or other undesired side-effects. Despite modification the viral vectors retain essential and identifiable components from the source virus. Such viral vectors generally retain the properties of being able to infect cells. They may be able to replicate in some (e.g. for manufacture), but not other, cells. This term does not include viral based plasmids or DNA, although such a plasmid may be used to create the viral vector.

Preferably the viral vector is an uncoated viral vector, i.e. a vector lacking an envelope. Adenovirus vectors are widely used to introduce foreign DNA into a wide variety of cells. For this purpose, various adenovirus vectors may be used, for example first, second and third generation adenovirus vectors may be used. These use various different vectors, e.g. lacking the early genes E1 and/or E3 or devoid of all viral coding sequences requiring the co-use of a helper virus (Ehrke-Schulz et al., 2016, supra). Adeno-associated virus (AAV) is also highly suitable as a gene therapy vector and methods for its production, which generally relies on the co-use of an Adenovirus or herpes simplex virus helper vector, an AAV Rep/Cap vector as well as the construct containing the transgene of interest (Grieger et al., 2016, supra). Such viruses have been used in the Examples and form preferred aspects of the invention, e.g. AAV type 2. Other viral vectors are also encompassed, such as modified vaccinia Ankara (MVA), but in a preferred aspect the viral vector is an adenovirus or an adeno-associated virus. Viruses of this sort which may have been modified for use as vaccines or for gene therapy are particularly preferred.

Release from Cell

As used herein “release” of the viral vector refers to the viral vector escaping from the confines of the cell in which it was produced such that it may be separated from that cell or from the remains of that cell. Preferably, substantially all of the produced viral vectors are released from the cell, or a significant majority (e.g. at least 40%, e.g. at least 50, 60, 70 or 75%, more preferably at least 80, 85, 90 or 95% of the viral vectors) are released. Although not wishing to be bound by theory, it is believed that the methods used herein allow pores to be made in the plasma membrane through which the viral vectors are released. Such pores are generated by the methods of the invention, as shown in the Examples. Such release may occur rapidly, but conveniently may be assessed or determined after 0-360 minutes, e.g. after 10-180 or 30-120 minutes.

As described hereinafter, the method may comprise additional steps of collection, purification, culture or production and thus the method may also provide a method of obtaining a preparation of VVs in accordance with such steps.

### Cell

The cell may be any cell which is able to produce the viral vector. Production of the viral vector refers to generation of the complete viral vector by the cell. This may result from infection of the cell with viral vectors in which case production involves replication of the viral vector. Alternatively, the cell may be transfected with polynucleotides (and optionally helper viruses) which provide the components necessary for production of the viral vector in that cell, i.e. provide all necessary material for the protein and nucleic acid components of the viral vector.

Conveniently the cell that is used is a cell that is known to be suitable for production of viral vectors, such as those described hereinafter. Conveniently the cell is a eukaryotic cell. In a preferred aspect, the cell is selected from mammalian cells, e.g. from humans or primates (such as HEK293, including HEK293T cells, and Vero cells which are commonly used for production of viral vectors) and insect cells such as sf9 and include engineered cells lines for viral vector production such as PER.C6.

The cells may be adherent or suspension cells.

### Photosensitising Agent

A “photosensitising agent” as referred to herein is a compound that is capable of translating the energy of absorbed light into chemical reactions when the agent is activated on irradiation at an appropriate wavelength and intensity to generate an activated species. The highly reactive end products of these processes can result in cyto- and vascular toxicity.

Photosensitizing agents may exert their effects by a variety of mechanisms, directly or indirectly. Thus, for example, certain photosensitising agents become directly toxic when activated by light, whereas others act to generate toxic species, e.g. oxidising agents such as singlet oxygen or other reactive oxygen species, which are extremely destructive to cellular material and biomolecules such as lipids, proteins and nucleic acids.

As described hereinbelow various photosensitising compounds may be used as the photosensitising agent. In the context of the present invention, it is likely that the VVs which may be collected by the methods of the invention, may be used therapeutically. Whilst only very small amounts of the photosensitising agent will be present in the final, purified VV formulation, using substances that are known to be safe in humans is a clear advantage. Several synthetic photosensitisers are already approved for use in human medicine (mainly based on porphyrin structures), but also many natural compounds or derivatives of natural compounds (e.g. chlorophyll derivatives; hypericin, curcumin) can be used as the photosensitising agent.

A range of such photosensitising agents are known in the art and are described in the literature, including in WO96/07432, which is incorporated herein by reference, and may be used in methods of the invention. There are many known photosensitising agents, including porphyrins, phthalocyanines and chlorins, (Berg et al., 1997, J. Photochemistry and Photobiology, 65, p 403-409, incorporated herein by reference). Other photosensitising agents include bacteriochlorins.

Porphyrins are the most extensively studied photosensitising agents. Their molecular structure includes four pyrrole rings linked together via methine bridges. They are natural compounds which are often capable of forming metal-complexes. For example in the case of the oxygen transport protein haemoglobin, an iron atom is introduced into the porphyrin core of heme B.

Chlorins are large heterocyclic aromatic rings consisting, at the core, of three pyrroles and one pyrroline coupled through four methine linkages. Unlike porphyrin, a chlorin is therefore largely aromatic, but not aromatic through the entire circumference of the ring.

For performance of the invention, conveniently the photosensitising agent localises to the plasma membrane. This allows direct action on that membrane and allows methods to be used which are not unnecessarily aggressive or disruptive to avoid contamination of the product to be collected. Photosensitising agents which localise to the plasma membrane are preferably amphiphilic or hydrophobic photosensitising agents. While hydrophobic compounds bind all membranes, amphiphilic compounds will initially anchor in the plasma membrane, and increasingly also in endosomes, as a result of invagination and internalisation of the plasma membrane during endocytosis. VVs that are not coated by a lipid membrane, including, but not limited to, adenovirus (AV) and adeno-associated virus (AAV), will not bind photosensitising agents that are amphiphilic or hydrophobic. Thus, by using amphiphilic or hydrophobic photosensitisers, production cells may be selectively disrupted by photochemical treatments without harming the VVs. Experiments have been conducted with both amphiphilic (e.g. TPCS₂ₐ and AIPcS₂ₐ) and hydrophobic photosensitisers (e.g. chlorin e6, verteporfin, temoporfin and protoporphyrin IX) and found to have similar effects. Whilst hydrophobic photosensitisers may locate to intracellular membranes other than the plasma membrane, even when using such photosensitisers no disruption of the nucleus is observed.

Amphiphilic photosensitisers (e.g. disulphonated photosensitising agents) include amphiphilic phthalocyanines, porphyrins, chlorins, and/or bacteriochlorins, and in particular include sulfonated (preferably disulfonated) meso-tetraphenyl chlorins, porphyrins, phthalocyanines and bacteriochlorins. In a preferred aspect, the photosensitising agent, preferably an amphiphilic or hydrophobic photosensitiser is selected from a porphyrin, phthalocyanine, purpurin, chlorin, benzoporphyrin, lysomotropic weak base, naphthalocyanine, cationic dye, tetracycline, or a derivative of any of said agents, preferably TPPS₄, TPPS₂ₐ, AIPcS₂ₐ or TPCS₂ₐ, or pharmaceutically acceptable salts thereof. Particularly preferred photosensitising agents are sulfonated aluminium phthalocyanines, sulfonated tetraphenylporphines, sulfonated tetraphenylchlorins and sulfonated tetraphenylbacteriochlorins. Also preferred are amphiphilic or hydrophobic chlorins (e.g. TPCS₂ₐ, temoporfin and chlorin e6), benzoporphyrins (e.g. verteporfin), porphyrins (e.g. protoporphyrin IX) and phthalocyanines (e.g. AIPcS₂ₐ). Particularly preferred are TPPS₂ₐ (tetraphenylporphine disulfonate), AIPcS₂ₐ (aluminium phthalocyanine disulfonate), TPPS₄ (meso-tetraphenylporphine tetrasulfonate), TPCS₂ₐ (tetraphenyl chlorin disulfonate) and TPBS₂ₐ (tetraphenyl bacteriochlorin disulfonate), or pharmaceutically acceptable salts thereof. Preferably the photosensitising agent is TPCS₂ₐ (Disulfonated tetraphenyl chlorin, e.g. Amphinex®) and/or a photosensitising agent used in the Examples.

As used herein “and/or” refers to one or both (or more) of the recited options being present, e.g. A and/or B includes the options i) A, ii) B or iii) A and B.

The structures of preferred photosensitising agents are provided below:

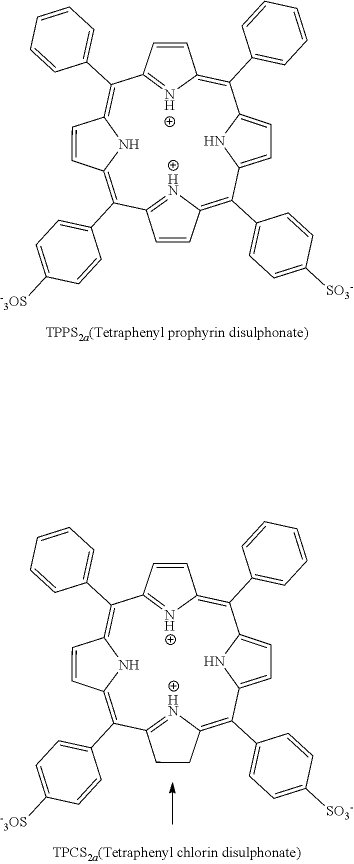

The arrow indicates the structural difference between the two molecules.

### Contacting Step

The cell is brought into contact with the photosensitising agent such that the photosensitising agent binds to the cell surface. Conveniently localisation to the plasma membrane is achieved, as discussed above. The photosensitising agent is used at an appropriate concentration and for an appropriate length of time to achieve this purpose. Whilst the method of the invention refers to a method performed on a cell, it will be appreciated that in practice multiple cells will be present and the description herein reflects that scenario.

The timing and concentration can easily be determined by a skilled person using routine techniques, and will depend on such factors as the particular photosensitising agent used, the irradiation to be used, the cells being irradiated and the arrangement of those cells. With regard to the latter, the cells may be in suspension or adherent and may be in layers or in other culture arrangements. Depending on that arrangement and the density of the cells different timings and doses may be necessary and may be varied or adjusted according to choice.

Conveniently, the contacting step a) is preferably performed for 0.5 to 120 minutes. The contacting step refers to the total contact time of the cell(s) with the photosensitising agent. That contacting time may be made up of a number of discrete separate contacting steps. As shown in the Examples, in methods of the invention genomic DNA is not released from the treated cells. Whilst not wishing to be bound by theory it appears that the nucleus remains intact, i.e. activation of the photosensitising agent does not disrupt the nuclear membrane sufficient to allow release of the genomic DNA. This is conveniently achieved with photosensitising agents that localise to the plasma membrane. In the alternative, the timing of the contacting step (and preferably also the concentration of the photosensitising agent and the time of irradiation) is selected to preserve the integrity of the nucleus (i.e. to avoid genomic DNA release) whilst disrupting the plasma membrane.

The agent may be removed from contact with the cell(s) for a period of time before the irradiation/illumination step. However, conveniently, illumination is performed immediately after the contacting step is completed, preferably with no intervening steps (e.g. washing).

As referred to herein the “contacting” refers to the time starting from the addition of the photosensitising agent to the first cell (where multiple cells are present). The end of the contacting step is the time at which irradiation is commenced or the photosensitising agent is removed (e.g. by removal of the liquid containing it and/or by washing the cells). This may mean that not all cells may be contacted with the photosensitising agent for the full duration of the contacting step, but the method is performed appropriately to ensure that all cells are contacted as evenly as possible to ensure maximal effects.

In a preferred aspect, the contacting step is from 2 to 30 minutes, for example for 5 to 15 minutes (or, 2 to 15, 5 to 30, 2 to 20 or 2 to 25 minutes). As noted above, to maximize efficacy all cells should be contacted with the photosensitising agent evenly and at the same time. When cells in culture are used, conveniently the cells are agitated to ensure mixing of the photosensitising agent with the cells and allow essentially simultaneous contact.

The concentration of the photosensitising agent is conveniently such that once taken up by the cell and activated by irradiation, the plasma membrane is lysed or disrupted. The Examples illustrate methods which achieve disruption of the plasma membrane and the generation of pores through which molecules or entities may pass. Similar experiments may be used to identify appropriate dosages for other systems involving different cells and/or photosensitising agents.

Conveniently, photosensitising agents as described herein may be used at a concentration of for example 0.01 μg/ml to 10 mg/ml, e.g. 0.1 to 500 μg/ml or 0.01 to 100 μg/ml, such as 0.1 to 50 μg/ml, e.g. from 0.01 to 10 μg/ml or 0.5 to 10 μg/ml. (Such ranges may apply, in particular, to each of the photosensitising agents used in the Examples, e.g. TPCS₂ₐ, verteporfin, chlorin e6, protoporphyrin and AIPcS₂ₐ at e.g., 0.1 to 500 μg/ml or 0.1 to 50 μg/ml and/or temoporfin at 0.01 to 100 μg/ml or 0.01 to 10 μg/ml.) The selection of the concentration is highly dependent on the system used, e.g. the photosensitising agent, the cell density and the intended irradiation protocol and should be selected accordingly.

Ideally the concentration of photosensitising agent used is as low as possible to avoid trace amounts of the photosensitising agent in the product and to avoid any damage to the VVs. Furthermore, since most photosensitisers are inactivated by irradiation (so-called photo-bleaching), their ability to induce toxic photochemical reactions can be inactivated shortly after, or even during, the method. Thus, the concentration of the photosensitising agent may be adjusted such that the irradiation dose sufficient to activate the photosensitising agent and disrupt the plasma membrane to allow release of the VVs also ensures that all of the photosensitising agent is photo-bleached at the end of the irradiation period. In that way, even if photosensitising agent remains in the final VV preparation, no photochemical reactions are possible in the recipient of the VVs, even if exposed to relevant light doses thereby avoiding light sensitivity.

The photo-bleaching properties may also be used to monitor the progress of the method and also to determine if activatable photosensitising agents remain. Photosensitising agents are usually fluorescent which is lost due to photo-bleaching. Thus, fluorescence may be monitored during the method, e.g. as a quality control analysis to ensure that activatable photosensitising agents have been removed from the final VV preparation.

### Irradiation

Once the photosensitising agent has been taken up by the cells, the cells may be irradiated with light of a wavelength effective to activate the photosensitising agent, wherein the irradiation is conducted at a dose of light and for a time sufficient to disrupt the plasma membrane of the cell, thereby releasing the viral vector.

“Irradiation” to activate the photosensitising agent refers to the administration of light directly or indirectly as described hereinafter. Irradiation may occur immediately after the contacting step, or may occur some time later, for example when the photosensitising agent is removed and cells washed and/or further cultured. When conducted after the contacting step, the cells may remain in contact with the photosensitising agent, but contact that follows irradiation is not considered part of the contacting step.

The light irradiation step to activate the photosensitising agent may take place according to techniques and procedures well known in the art. The dose, wavelength and duration of the illumination must be sufficient to activate the photosensitising agent, i.e. to generate reactive species.

The wavelength of light to be used is selected according to the photosensitising agent to be used. Conveniently, to aid ease of use, visible light (400-700 nm) may be used, e.g. normal indoor light. In the alternative, suitable artificial light sources are well known in the art, e.g. using blue (400-475 nm) or red (620-750 nm) wavelength light. For TPCS₂ₐ, both blue and red light may be used. For example, a wavelength of between 400 and 500 nm, more preferably between 400 and 450 nm, e.g. from 410-430 nm or 430-440 nm, and even more preferably approximately 435 nm (or 425 nm), or 435 nm (or 425 nm) may be used. In the alternative light with a wavelength of between 630 and 675 nm may be used, e.g. from 645-660 nm, e.g. 652 nm. Where appropriate the photosensitiser, e.g. a porphyrin or chlorin, may be activated by green light, for example the KillerRed (Evrogen, Moscow, Russia) photosensitiser may be activated by green light.

Suitable light sources are well known in the art, for example the LumiSource® lamp of PCI Biotech AS. Alternatively, an LED-based illumination device which has an adjustable output power of up to 60 mW and an emission spectrum of 430-435 nm (or 410-430 nm) may be used. For red light, a suitable source of illumination is the PCI Biotech AS 652 nm laser system SN576003 diode laser, although any suitable red light source may be used.

The cells may be in various types of vessels (or containers) during photochemical treatments, ranging from small tubes, plastic vessels, bags, glass containers, to large metal tanks. Throughout, the cells will be in aqueous solutions (cell medium, buffers, etc.) that are penetrable by most light wavelengths. For very large bioreactors, dark vessels, or strongly coloured solutions, blue light can be applied, which penetrates very well through aqueous solutions.

Vessels or containers used to handle the production cells may be made of plastic, glass, or metal. Relatively small plastic and glass vessels may be used and are preferably translucent, such that irradiation can be achieved by placing the light source in proximity. Larger vessels, such as bioreactor tanks, may be made of metal. For these, the light source is conveniently inside the vessel in closed processes, or may be placed outside/on top of the vessel in open processes. Given the limited size of these vessels, one or a few light sources should be sufficient. In addition, additional covers with reflecting materials to ensure even irradiation may be used.

The time for which the cells are exposed to light in the methods of the present invention may vary. Ideally in methods of the invention the host cell (production cell) is disrupted sufficiently to release the VVs but damage beyond that which is necessary for this purpose is avoided.

A preferred length of time for the irradiation step depends on factors such as the target cell, the photosensitiser, the amount of the photosensitiser accumulated in the target cells, the overlap between the absorption spectrum of the photosensitising agent and the emission spectrum of the light source and the cell density. Generally, the length of time for the irradiation step is in the order of seconds to minutes or up to several hours (even up to 12 hours), e.g. preferably up to 180 minutes e.g. from 30 seconds to 120 minutes, preferably 1 to 30 minutes, preferably for 2 to 10 minutes.

Conveniently, when cells in suspension are used, the cells are agitated during the irradiation step to ensure that photosensitising agent is mixed with, and has good access to, each cell. Mixing may be achieved by any appropriate means including rocking, stirring, shaking and rotation.

Appropriate light doses can be selected by a person skilled in the art and again will depend on the photosensitiser used and its concentration and the amount of photosensitiser accumulated in the target cell(s). It will also depend on the arrangement, density and total amount of the cells to be irradiated.

The cells producing the VV may be grown on a surface (adherent) or in suspension. Adherent cells may be grown in stacks. Consequently, cells may be exposed to different light doses if a single light source is used. The light dose is selected to ensure that the majority of cells are exposed to a light dose sufficient to achieve the desired effect, i.e. disruption of the plasma membrane. Higher density, and higher total amounts of cells may require a higher light dose to achieve the same effects.

The following description is provided for adherent cells which form a single layer of cells but may be scaled accordingly for different cell arrangements.

The light doses are usually lower when photosensitisers with higher extinction coefficients of the visible spectrum are used (e.g. higher extinction coefficients in the red area, or blue area if blue light is used, depending on the photosensitiser used). For example, if the LumiSource® lamp (blue light) is employed a light dose in the range of 1-6 J/cm² at a fluence range of 0.1-20 (e.g. 13 as provided by Lumisource®) mW/cm² may be appropriate.

As noted above the irradiation step is used to disrupt the plasma membrane. As referred to herein “disruption” of the plasma membrane refers to destruction of the integrity of that membrane to allow the flow of molecules or entities into or out of the cell which is not possible when the membrane is intact. Flow into or out of the cell refers to flow between the exterior and interior of the cell (and/or vice versa), i.e. not bounded by the plasma membrane or other intra- or extra-cellular cell-derived membrane. Thus, disruption of the plasma membrane does not extend to the formation of blebs (bulges in the plasma membrane) which may detach to become extracellular vesicles.

In accordance with methods of the invention, the membrane is disrupted to the extent that VVs can pass from the inside of the cell to its exterior. The cell may have a disrupted membrane that only allows VVs and smaller molecules/entities to be released, e.g. through pores, or may be more extensively disrupted, e.g. the cell may be lysed. A “pore” refers to a hole in the plasma membrane connecting the interior of the cell to the exterior environment in which the cell is present and through which molecules/entities may flow.

Preferably, substantially all of the cells, or a significant majority (e.g. at least 40%, e.g. at least 50, 60, 70 or 75%, more preferably at least 80, 85, 90 or 95% of the cells) are killed (of those subject to the treatment). Cell viability following the treatment can be measured by standard techniques known in the art such as the MTS test, as illustrated in the Examples.

As described above, methods of the invention disrupt the plasma membrane of a cell to allow release of the viral vectors, e.g. through pores. Methods of the invention, as illustrated in Example 6, do not form extracellular vesicles. Furthermore, no blebbing on the cells' surfaces is observed. Thus, methods of the invention are such that preferably extracellular vesicles are not formed.

### Release of Cellular DNA

As discussed above, the methods of the invention have the advantage that cellular DNA may be retained in the cell whilst the VV are released after illumination. The conditions of photochemical lysis are preferably selected accordingly to achieve this outcome. Thus in a preferred aspect cellular DNA, in particular genomic DNA, remains in said cell after the disruption of said plasma membrane. Preferably, substantially all of the cellular DNA (in particular genomic DNA), or a significant majority (e.g. at least 30%, e.g. at least 40, 50, 60, 70 or 75%, more preferably at least 80, 85, 90 or 95% of the cellular DNA) remains in the cell. The genomic DNA remains in the cell after release of the viral vector and is conveniently assessed or determined after 0-360 minutes, e.g. after 10-180 or 30-120 minutes. Thus in preferred aspects at least 30% (or higher as described above) of the genomic DNA in said cell prior to illumination remains in said cell after release of said viral vector.

Retention of the DNA is achieved when the plasma membrane is disrupted by the generation of pores in said membrane which prevent the release of the cellular DNA.

### Other Reagents Used During Lysis Step

As shown in the Examples, the method described herein disrupts the plasma membrane such that viral vectors may be released from the production cell. Whilst this may be used as the sole means to release the viral vectors, additional methods to achieve release may also be used. Thus for example, a lysing agent may be added to the cell in step a), b) and/or c). (However, in some embodiments disruption of the plasma membrane is achieved solely by the photochemical lysis method of the invention and/or no lysing agent is employed in the method of the invention.) A “lysing agent” is any agent which is capable of lysing the cell used in the method under the conditions used. Such a lysing agent may be a detergent or hypotonic buffer or enzymes for that purpose. In the alternative other physical steps of lysis may be used such as freeze-thawing or sonication.

The lysing agent may be added before, during and/or after the contacting and irradiation step to aid disruption of the plasma membrane by the photosensitising agent. Conveniently the steps are combined, for example the photochemical treatment is performed at the same time as the lysing agent is added, e.g. the photosensitising agent is added in a detergent-based lysis buffer. The co-use of methods of lysis/plasma cell disruption is expected to simplify downstream purification steps or provide improved VV preparations due to higher VV purity early in the method. However, as noted above, the method of the invention allows release of VVs without any additional lysis methods. In this scenario DNA leakage can be avoided. If additional lysis methods are used, whilst this may result in superior release of the VVs, DNA leakage may occur. The method to be used should therefore be selected accordingly depending on the importance of VV release versus DNA leakage.

### Collection and Purification

Once the VVs have been released from the cell they may be collected. This may occur after in line with the timing of release as discussed hereinbefore, conveniently after 0-360 minutes, e.g. after 10-180 or 30-120 minutes. As referred to herein “collection” refers to separation from other components of the source (production) cells. Collection does not necessarily entail purification though the process of collection may increase the purity of the VVs. Further purification steps following collection may be performed.

Any convenient collection methods may be used depending on how the irradiation step was conducted. Conveniently however, the collection is achieved by removal of cell debris. Such cell debris includes whole cells (which may result if the irradiation step did not result in cell lysis) or lysed cells and the debris released from such cells. Depending on the mixture from which VVs are to be collected, different collection techniques may be used.

Although the irradiation step may be conducted in different ways, the cells are likely to be surrounded by at least some aqueous medium. As discussed in more detail hereinafter, in some cases only a minimum aqueous medium may be present, e.g. when the majority of the culture medium has been removed after culture and before irradiation and/or the cells have been washed after irradiation. However, in other cases the irradiation may be conducted when considerably more aqueous medium is present, e.g. containing the photosensitising agent and/or lysing agents.

Thus, in a preferred embodiment the cell(s) is in an aqueous medium during steps a) and b). The aqueous medium is preferably cell culture medium and/or a solution containing the photosensitising agent and/or lysing agent when present. In that case collection is performed by separation of the aqueous medium from cell debris which is not suspended in the medium. Conveniently this separation may be performed by centrifugation or filtration. However, in some cases, depending on the extent of lysis of the production cells, it may be possible to collect the VVs by collection of the aqueous medium into which they were released as a supernatant to essentially intact production cells.

Where a cell culture medium is used as the aqueous medium, that medium may be serum-free or serum-supplemented.

The VVs once collected may be further purified. Conveniently techniques known in the art for this purpose may be used. Thus, in a preferred aspect, at least one of the following methods selected from centrifugation (e.g. density centrifugation), sonication, freeze-thawing, enzyme digestion and liquid chromatography, is used. Greiger et al., 2016, supra and Ehrke-Schulz et al., 2016, supra describe suitable purification techniques for AAV and adenovirus, respectively. Conveniently a purity of at least 50% (w/w, dry weight), preferably at least 60, 70, 80, 90, 95 or 99% w/w (dry weight), is achieved.

The yield of VVs may be assessed in a number of ways including OD titre or qPCR. The latter may be used to identify and quantify infectious particles. Preferably a yield of at least 1×10⁵ vector genome containing particles (vg)/cell or at least 1×10¹⁰ vg/l, 2×10¹⁰ vg/l, 1×10¹¹ vg/l or 1×10¹² vg/l of cell culture is achieved.

### Methods of Culture and Release

The method of the invention may also be performed in which prior to contacting step a) a step in which said viral vector is produced in the cell is performed. Preferably the viral vector is produced by culturing the cell to produce the viral vector and optionally the culture supernatant is removed before step a).

When a VV production step is included as part of the method, the present invention provides a method of releasing a viral vector from a cell in which said viral vector has been produced, comprising:
a) producing said viral vector in said cell, preferably by culturing said cell to allow said viral vector to be produced by said cell and optionally removing the culture supernatant before step b),
b) contacting said cell in which said viral vector is present (has been produced) with a photosensitising agent,
c) irradiating said cell with light of a wavelength effective to activate said photosensitising agent, wherein said irradiation is conducted at a dose of light and for a time sufficient to disrupt the plasma membrane of said cell, thereby releasing said viral vector, and
d) optionally collecting and/or purifying said released viral vector.

All of the definitions and preferred embodiments described hereinbefore similarly apply to this method.

In production step a), the viral vector is produced in the cell. Thus, the cell is infected or transfected with the relevant components necessary to produce the viral vector and the viral vector is amplified or generated in that cell. Conveniently this is achieved by culturing the host cells under conditions that allow production of the VVs. Appropriate conditions for this purpose are well known and dependent on the cell to be used as the producing cell (or host) and the VV to be produced. Cell cultures may be in suspension or on plates or other solid supports if adherent cells are grown.

The production step is continued until VVs are present in the cells in appropriate numbers, e.g. at least 1×10⁵ vector genome containing particles (vg)/cell. This may take from 24 hours to 7 days, e.g. 2-5 days. Cells exhibiting clumping are generally ready for harvesting.

Once the production step has been completed the production cells containing VVs are prepared for application of the photosensitising agent and irradiation.

Conveniently the medium (e.g. supernatant) in which the cells are present may be removed. The cells may be washed. The photosensitising agent and optionally lysing agent may be added. In the alternative the photosensitising agent and optionally lysing agent may be added to the medium in which the cells have been grown. The cells may be washed after this step once the photosensitising agent has been taken up before irradiation or irradiation may be performed without a washing step. Thus in alternative aspects of the invention, cells are washed after completion of the contacting step with the photosensitiser and before irradiation, or, cells are not washed at that time.

### Methods of Transfection/Infection, Culture and Release

The method of the invention may also be performed in which prior to production step a) a step is performed in which the cell is infected with one or more of the viral vectors or transfected with one or more polynucleotides which allow the production of the viral vector in the cell. This step provides a competent producing cell. This may be performed by techniques known in the art (see e.g. Greiger et al., 2016, supra and Ehrke-Schulz et al., 2016, supra). In one alternative, the cell is transfected or infected with one or more polynucleotides and/or virus vectors which allow production of the viral vector. These polynucleotides may be plasmids or other polynucleotides. Viral vectors may be provided where necessary, e.g. in the form of a helper virus. This is generally performed as a pre-amplification step to obtain viral vectors for infection of cells. However, in both cases the methods of the invention may be used to release the VVs.

When the steps of infection/transfection of a cell and a VV production step is included as part of the method, the present invention provides a method of releasing a viral vector from a cell in which said viral vector has been produced, comprising:
a) infecting a cell with one or more viral vectors and/or transfecting said cell with one or more polynucleotides which allow the generation of said viral vector in said cell,
b) producing said viral vector in said cell, preferably by culturing said cell to allow said viral vector to be produced by said cell and optionally removing the culture supernatant before step c),
c) contacting said cell in which said viral vector is present (has been produced) with a photosensitising agent,
d) irradiating said cell with light of a wavelength effective to activate said photosensitising agent, wherein said irradiation is conducted at a dose of light and for a time sufficient to disrupt the plasma membrane of said cell, thereby releasing said viral vector, and
e) optionally collecting and/or purifying said released viral vectors.

All of the definitions and preferred embodiments described hereinbefore similarly apply to this method.

As referred to herein “infection” refers to the viral vector being taken up into the cell via its normal mode of transport. Generally, the viral vector replicates within the cell to provide multiple copies of itself, though this may require assistance, e.g. from a helper viral vector. Transfection refers to the take-up of polynucleotides by non-viral methods.

The methods of the invention and their various steps may be performed in a variety of different ways, some of which are described hereinbefore.

By way of one example, after the production/culture step the cells are collected and centrifuged. The supernatant is discarded. Photosensitising agent is added in solution to the cells. Other lysing agents may be added, e.g. detergent. The cells are allowed to take up photosensitising agent whilst mixing. The solution is then irradiated. Conveniently the cells are in a translucent container so that light readily passes into the container and to the cells. Natural light may be used. Following release from the cells the VVs may be purified by normal downstream processing steps such as centrifugation, sonication, freeze-thawing, enzyme digestion (e.g. with DNase) and/or liquid chromatography. Two or more of the steps may be performed in the same container. For example, the production step and the photochemical treatment step may be performed in the same container (e.g. the culture vessel) or they may be performed in separate containers. Such a scenario may be considered performing the photochemical treatment “upstream”. Similarly, the photochemical treatment step may be performed in the same container as used for subsequent purification steps, e.g. in a sonication vessel or tube. This may be considered a “downstream” photochemical treatment, i.e. after the cells have been collected from the culture vessel.

Preparation of VVs obtainable or obtained by method of the invention form further aspects of the invention.

### Kit/Apparatus

Furthermore, the invention provides a kit for use in methods of the invention. Thus, in a further aspect the invention provides a cell harvesting kit or apparatus for releasing a viral vector from a cell comprising:
a) a photosensitising agent; and
b) a light source to irradiate said cell.

The photosensitising agent is as described hereinbefore. The light source is suitable for irradiation as described hereinbefore. The light source to be provided is selected based on its intended use and in particular the form in which the cells are presented, the photosensitising agent to be used and its concentration.

The kit or apparatus may additionally comprise a container in which the cells may be contained. This container may be used solely for the purpose of the irradiation step and subsequent steps, and/or may be used for initial culture steps. Thus, in a preferred aspect the container is suitable for cell culture and/or purification of the cells. As described hereinbefore the culture and irradiation steps may be performed in a variety of different vessels or containers. By way of example the container may be a plate (e.g. stack of plates/cell factory), tank or bag (e.g. a bioreactor bag), which is preferably translucent. Conveniently however, the container is a bag or a tank and/or the light source is attached to the container.

To aid uniformity during the method, the kit or apparatus may additionally comprise a means to agitate the cells. This may be provided as part of the container or may be provided separately to be placed inside or outside the container to agitate the cells directly or indirectly, respectively. By way of example a cell culture rocker or a stirrer within a container may be provided.

Additional components may also be provided, including e.g. culture medium, and reagents for lysis, washing buffers and solutions. Optionally said kit may also contain a package insert describing how the method of the invention should be performed.

The kits and apparatuses of the invention may be used in methods of the invention.

The methods described in the Examples form further preferred aspects of the invention. All combinations of the preferred features described above are contemplated, particularly as described in the Examples. The invention will now be described in more detail in the following non-limiting Examples with reference to the following drawings in which:

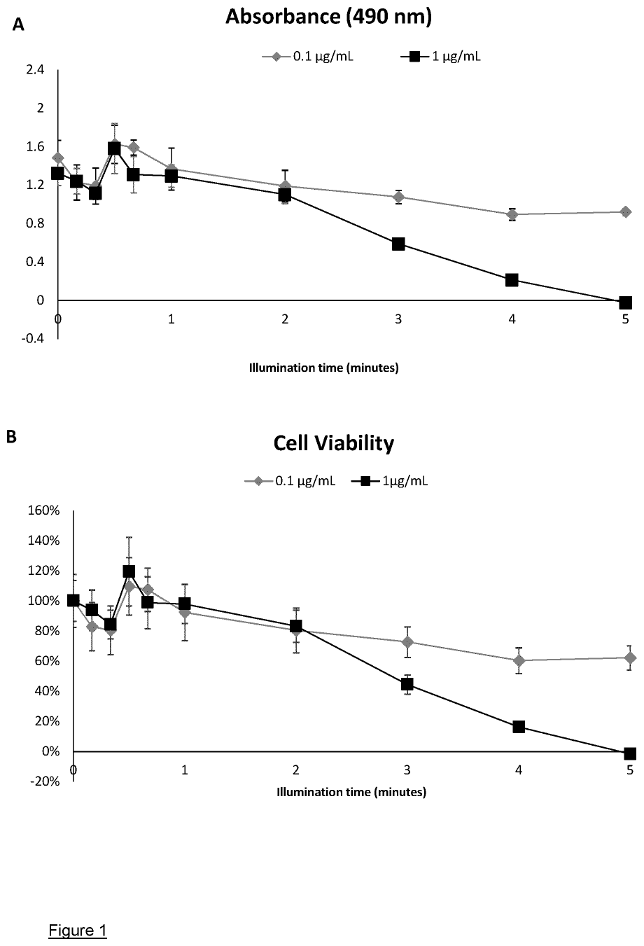

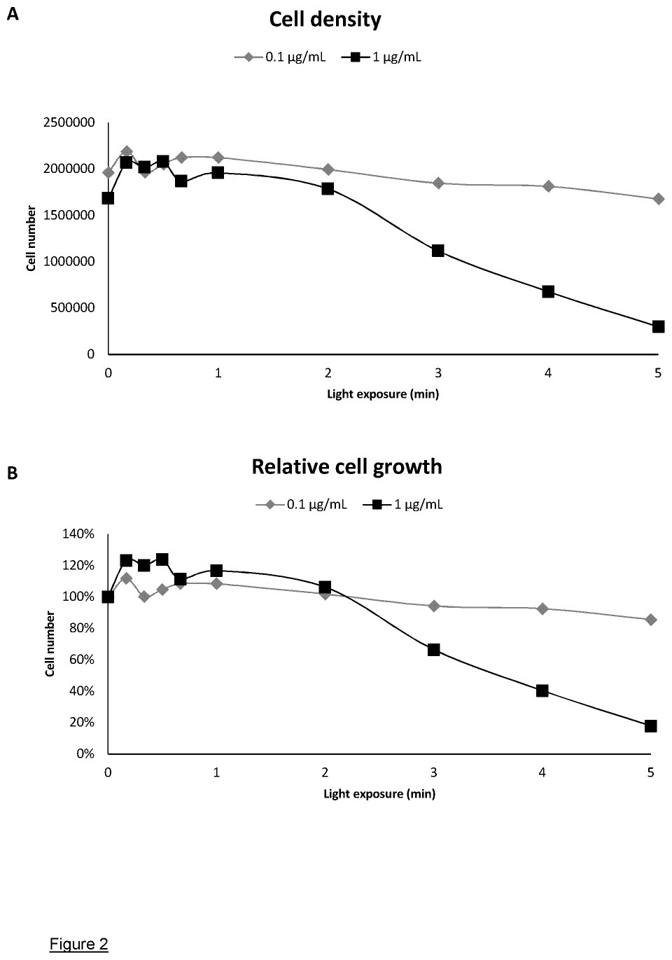

FIG. 1 shows the results of an MTS assay performed on Jurkat cells treated with TPCS₂ₐ at 0.1 ug/mL or 1 ug/mL for 10 minutes followed by irradiation for the times indicated and incubation for 48 hours. A) shows absorbance dependent on time of irradiation and B) shows the corresponding viability of the cells.

FIG. 2 shows the results of a cell counting experiment on Jurkat cells treated with TPCS₂ₐ at 0.1 ug/mL or 1 ug/mL for 10 minutes followed by irradiation for the times indicated and incubation for 48 hours. A) shows cell density dependent on time of irradiation and B) shows the corresponding relative cell growth for the same irradiation times.

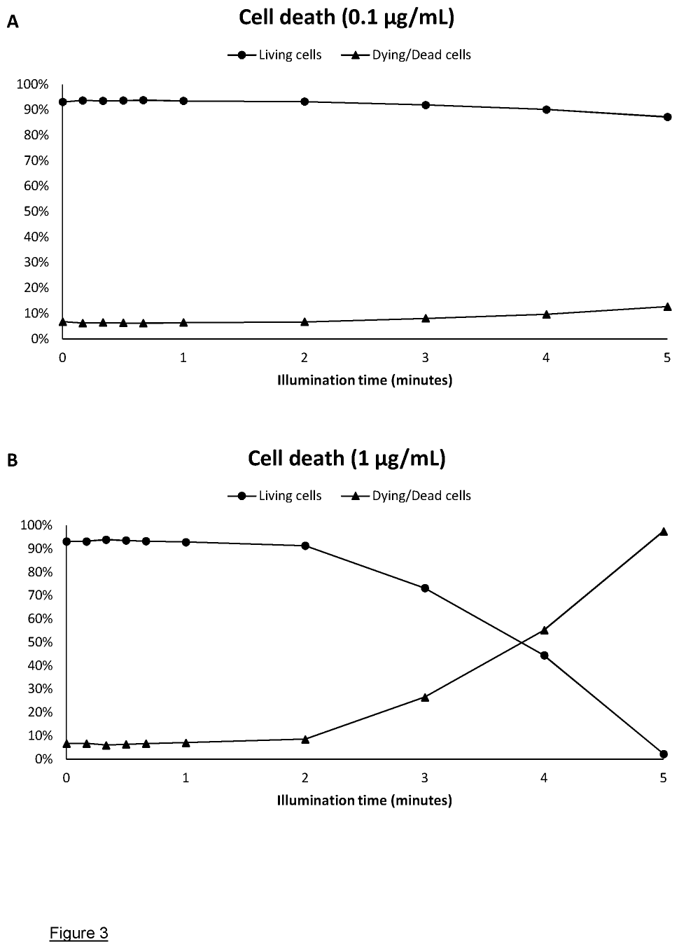

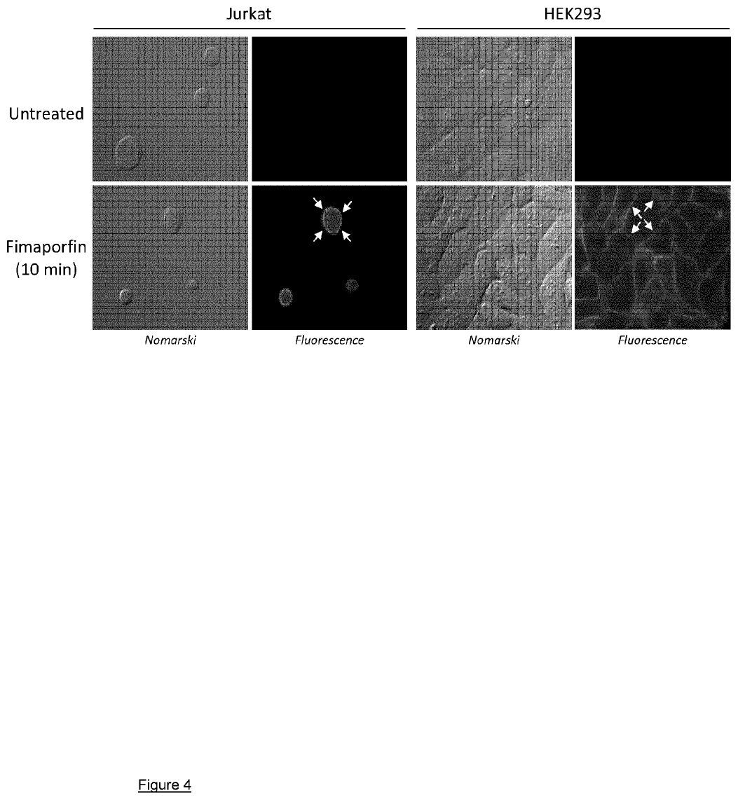

FIG. 3 shows cell death (based on Hoechst 33258 staining and flow cytometry) of Jurkat cells treated with TPCS₂ₐ at 0.1 ug/mL (A) or 1 ug/mL (B) for 10 minutes followed by irradiation for the times indicated and incubation for 48 hours.

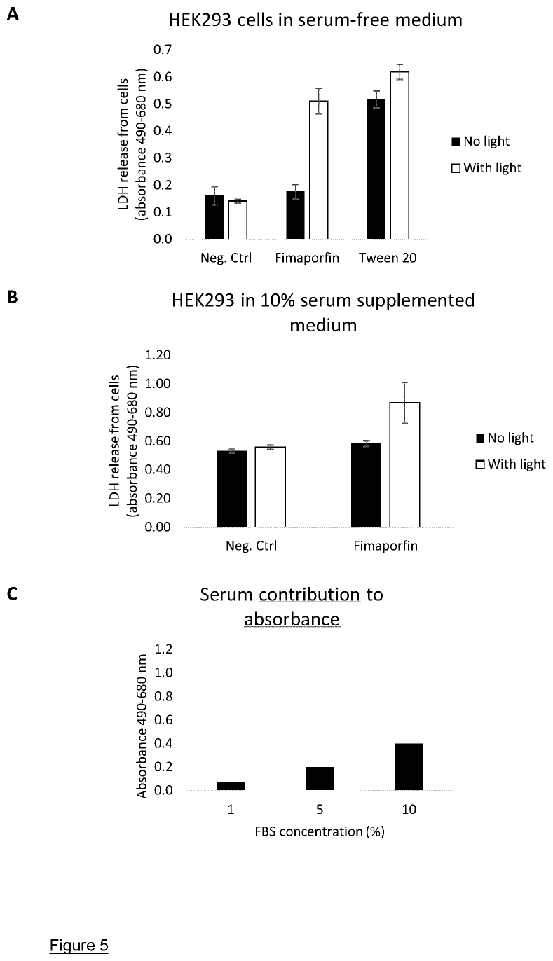

FIG. 4 shows the cellular localisation of TPCS₂ₐ at the plasma membrane of Jurkat cells treated with TPCS₂ₐ at 1 μg/mL in complete RPMI 1640 and HEK293 cells treated with TPCS₂ₐ at 5 μg/mL in DMEM medium for 10 minutes. White arrows indicate the presence of TPCS₂ₐ at the plasma membrane.

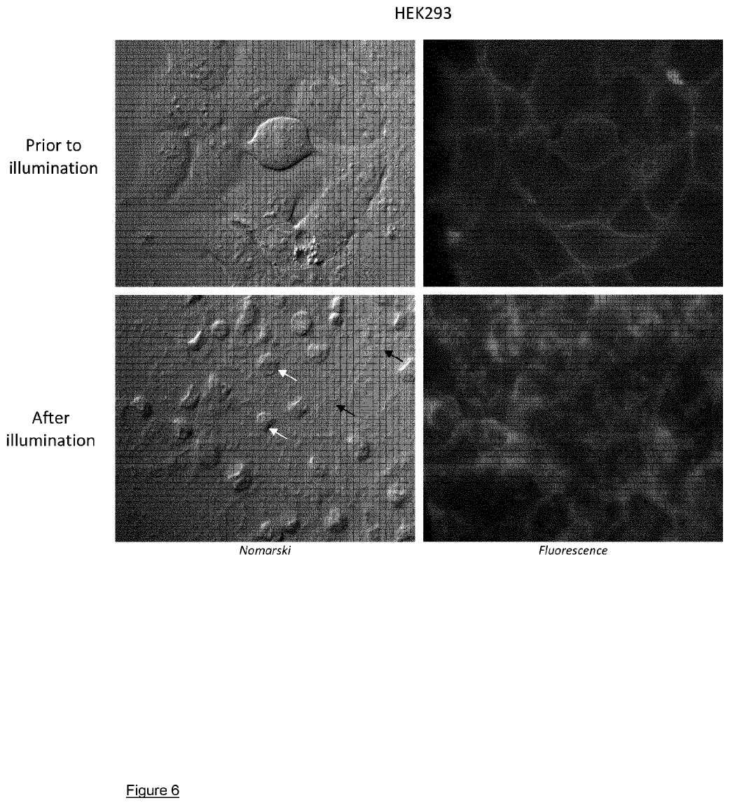

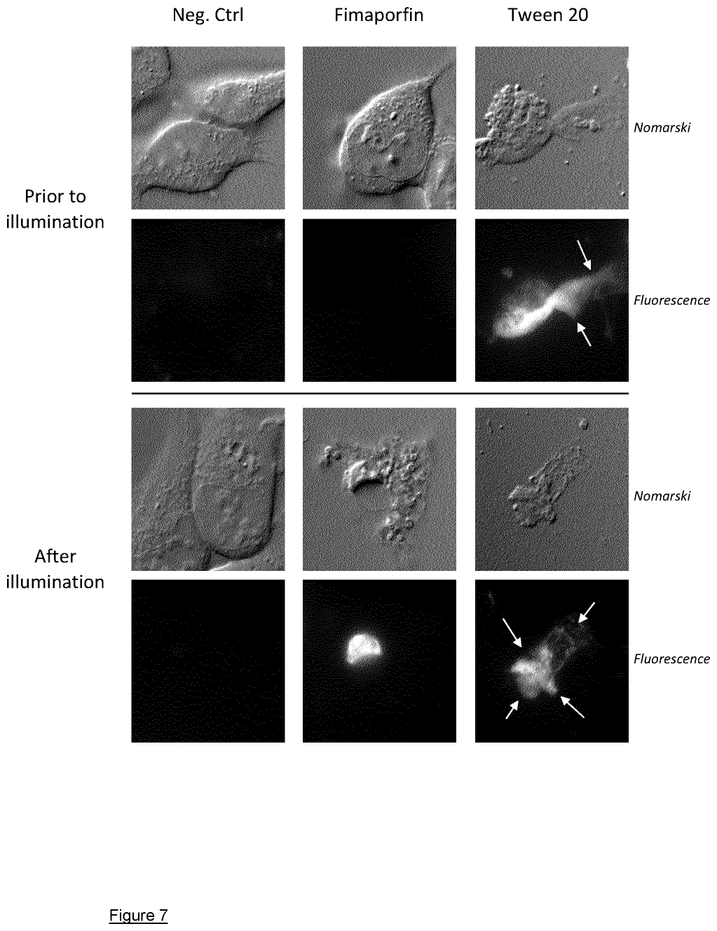

FIG. 5 shows the results of LDH assays to assess the leakage of cytosolic material from HEK293 cells treated with TPCS₂ₐ at 0.5 μg/mL in serum-free DMEM or Tween 20 at 0.5% (A), or TPCS₂ₐ at 50 μg/mL in 10% serum DMEM (B) for 10 minutes followed by irradiation (“with light”) for 5 minutes or no irradiation (“no light”). LDH release was assessed after 2 hours. (C) shows the background absorbance values of the LDH assay at FBS concentrations of 1%, 5% and 10%.

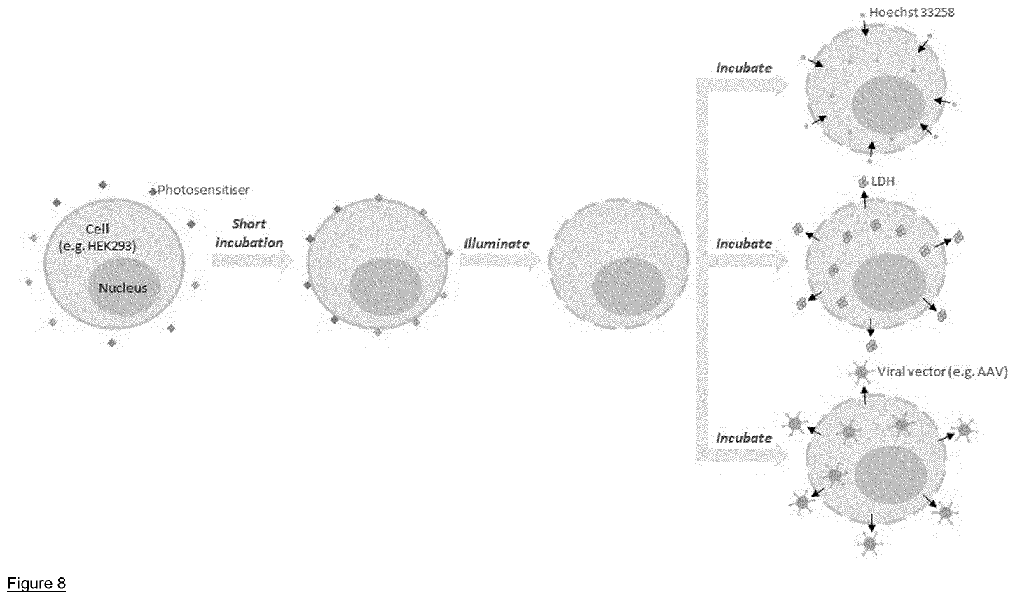

FIG. 6 shows the changes to cellular morphology in HEK293 cells treated with TPCS₂ₐ at 5 μg/mL in complete DMEM for 10 minutes, followed by irradiation for 5 minutes. The figure shows cells which have been imaged by light (Nomarski) and fluorescence microscopy prior to illumination and 2 hours after illumination. White arrows indicate cell corpses that remain after illumination and black arrows indicate cell debris after illumination.

FIG. 7 shows the impact of photochemical treatment or detergent lysis on cellular morphology and DNA leakage in HEK293 cells treated with TPCS₂ₐ at 5 μg/mL or Tween 20 at 0.5% in complete DMEM for 10 minutes, followed by irradiation for 5 minutes. Hoechst 33258 was added to samples 2 minutes prior to imaging. The figure shows cells which have been imaged by light (Nomarski) and fluorescence microscopy prior to illumination and 2 hours after illumination.

FIG. 8 is a diagram illustrating how photochemical treatments may be employed to selectively permeabilise the plasma membranes of producer cells to release viral vectors without DNA contamination or leakage (referred to herein as photochemical lysis, PCL).

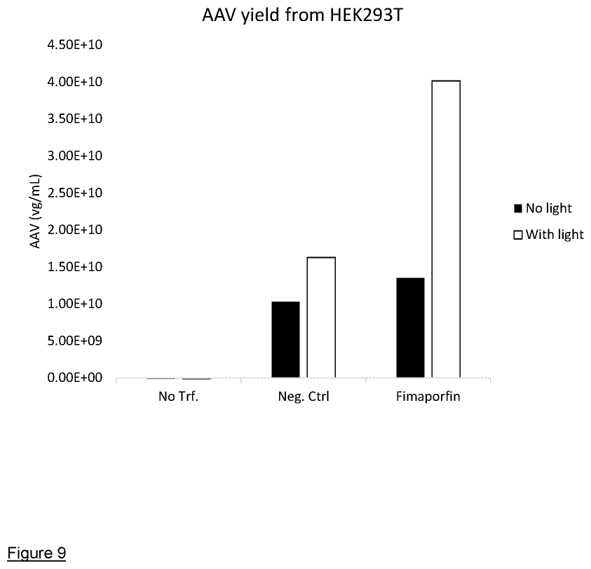

FIG. 9 shows the use of photochemical lysis to release viral vectors (AAV serotype 2/AAV2) from producer cells (HEK293T adherent cells). The figure shows untransfected cells (No Trf.), transfected cells not treated with photosensitiser (Neg. Ctrl) and transfected cells treated with photosensitiser (Fimaporfin, TPCS₂ₐ). Cells were irradiated with blue light as indicated. The figure is representative of three independent experiments.

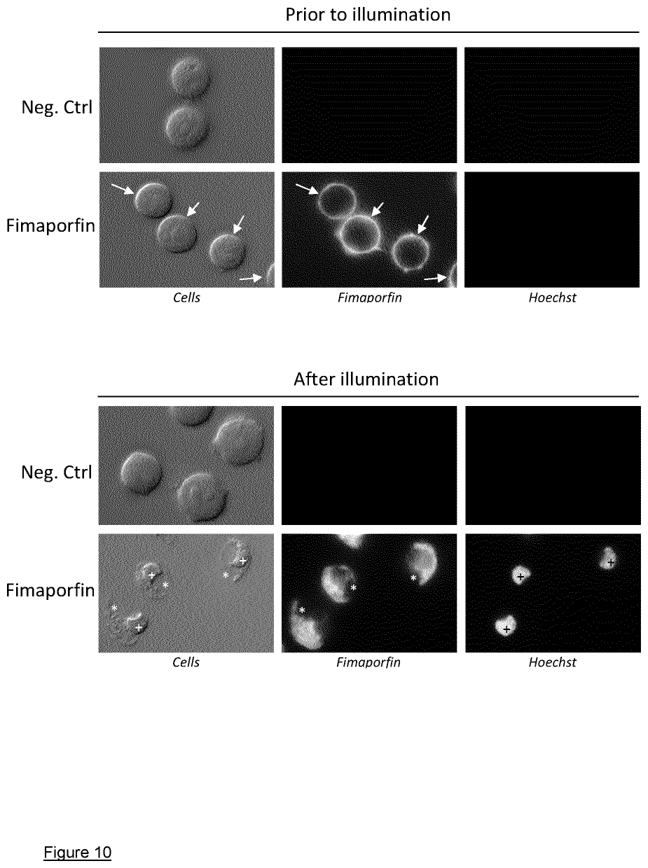

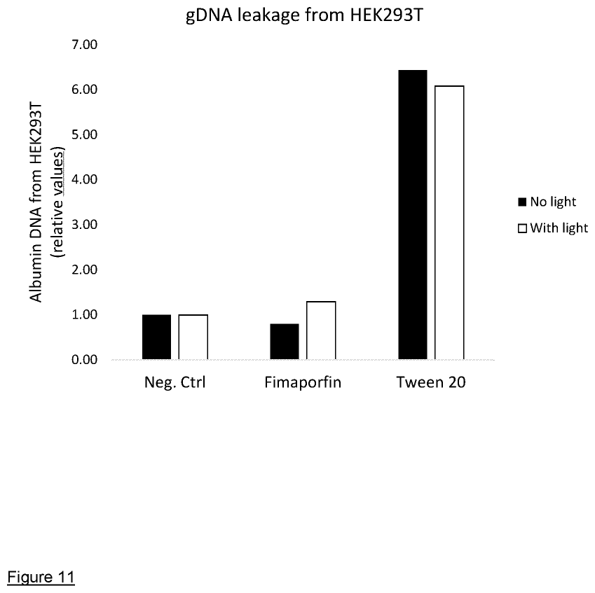

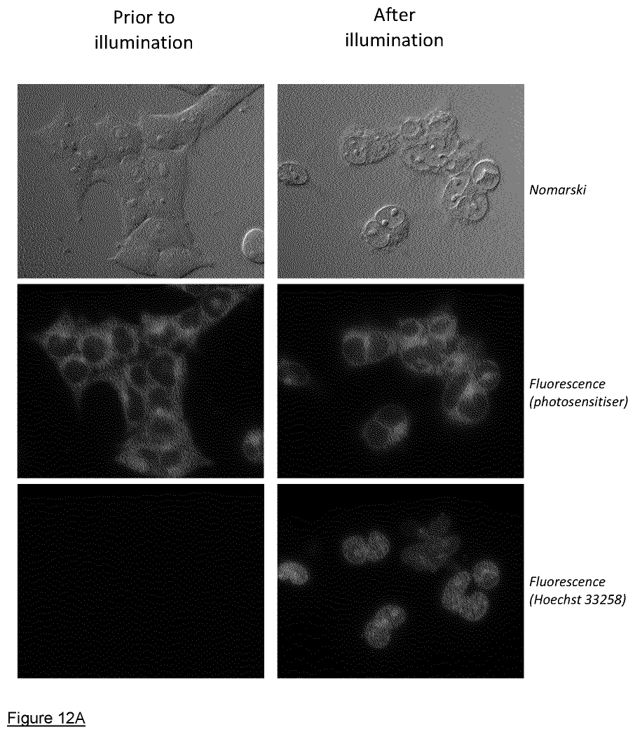

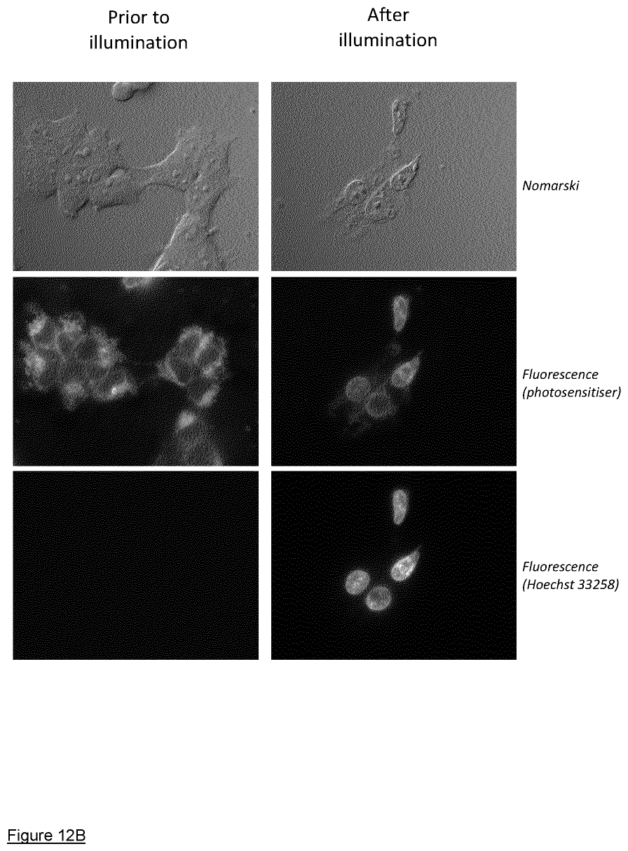

FIG. 10 shows the impact of photochemical lysis on HEK293T suspension cells. HEK293T suspension cells were treated with fimaporfin or fimaporfin solvent without photosensitiser (“Neg. Ctrl”), followed by blue light illumination. Hoechst 33258 DNA stain was used to stain free DNA or DNA in cells with plasma membranes. Cells were imaged by light (Nomarski) microscopy and fimaporfin and Hoechst fluorescence by fluorescence microscopy after 10 minutes (i.e. prior to illumination) and 2 hours after illumination. Arrows indicate the plasma membrane localisation of fimaporfin. Asterisks indicate where the plasma membrane has been disrupted and the corresponding absence of fimaporfin staining. Plus signs indicate Hoechst positive DNA and the corresponding localisation of DNA in lysed cell corpses.

FIG. 11 shows the impact of photochemical lysis on genomic DNA leakage in HEK293T cells. HEK293T cells were treated with 5 μg/mL TPCS₂ₐ (fimaporfin) or 0.5% Tween 20 in complete DMEM for 10 minutes, followed by blue light illumination for 5 minutes (“With light”) or no illumination (“No light”). “Neg. Ctrl” refers to cells that received fimaporfin solvent but no photosensitiser, to control for solvent effects on cell lysis. After 2 hours incubation at 37° C. 5% CO₂, supernatants from all samples were collected. Genomic DNA in supernatants was analysed by ddPCR using primers targeting the human albumin gene. Values were normalised to “Neg. Ctrl”.

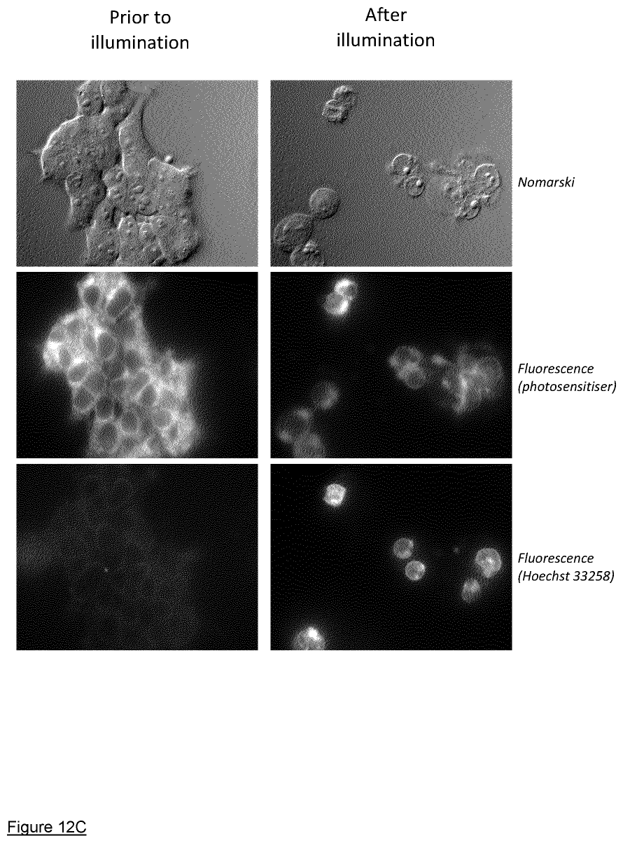

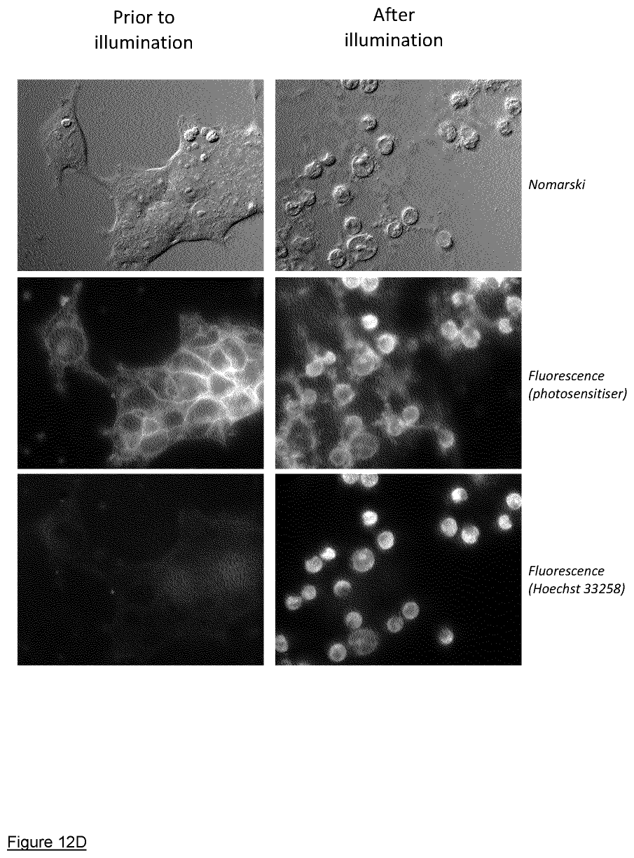

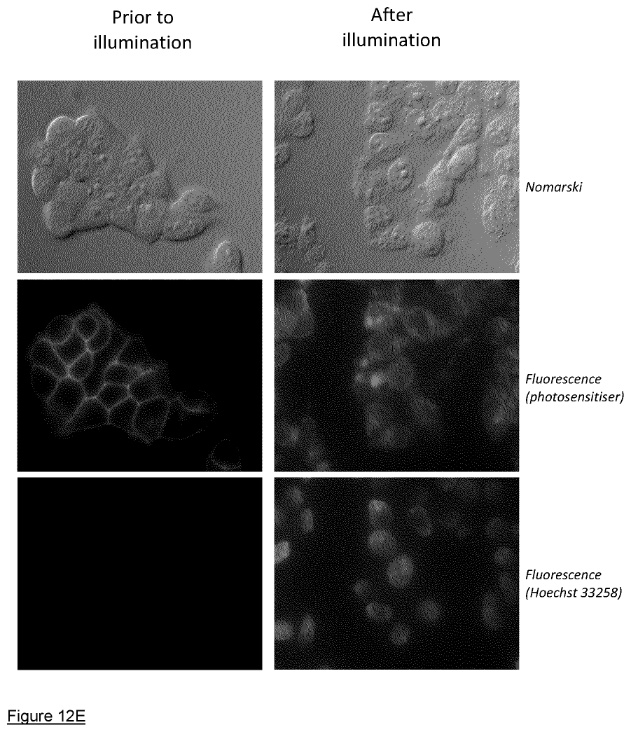

FIG. 12 shows the impact of photochemical treatments on cellular morphology and DNA leakage in HEK293T cells treated with 5 μg/mL verteporfin (FIG. 12A), 0.03 μg/mL temoporfin (FIG. 12B), 3 μg/mL chlorin E6 (FIG. 12C), 30 μg/mL protoporphyrin IX (FIG. 12D), or 10 μg/mL AIPcS₂ₐ (FIG. 12E) in complete DMEM for 10 minutes, followed by irradiation for 5 minutes. Hoechst 33258 was added to samples 2 minutes prior to imaging. The figure shows cells which have been imaged by light (Nomarski), and Hoechst and photosensitiser fluorescence imaged by fluorescence microscopy prior to illumination and 2 hours after illumination.

### EXAMPLE 1: GENERATION OF PORES IN JURKAT CELLS BY IRRADIATION AFTER TPCS_{2A} TREATMENT

### Materials and Methods

Jurkat cells were incubated with 0.1 ug/mL or 1 ug/mL fimaporfin (TPCS₂ₐ, tetraphenyl chlorin disulfonate) for 10 minutes in complete RPMI 1640 medium. The cells were subsequently illuminated for various durations with blue light as indicated in the figures. Following irradiation, the cells were incubated for 48 hours at 37° C. and 5% CO₂, after which several analyses of cell health were performed.

Blue light irradiation/illumination was performed using LumiSource according to the manufacturer's protocol (PCI Biotech)

MTS metabolism was performed as a measure of metabolic activity and performed according the manufacturer's protocol (Promega).

Cell counting was performed as a measure of cell growth using a Coulter Counter by Beckman Coulter according to the manufacturer's protocol. Counting was performed 48 hours after irradiation.

Entry of Hoechst 33258 (Thermo Fisher Scientific) into cells was performed as a measure of cell death. When Hoechst 33258 enters cells, it binds to double-stranded DNA generating a strong fluorescent signal. Entry of the stain into the cells requires the presence of pores in the plasma membrane and is a late-stage measure of cell health, i.e. cell death. Hoechst 33258 staining and flow cytometry was performed according to the manufacturer's protocol (Thermo Fisher Scientific).

### Results

MTS provides an assay based on reduction of an MTS tetraxolium compound by cells to produce a detectable dye. This production reduces as metabolic activity of the cells decreases and is an indicator of viability. FIG. 1 shows that on irradiation absorbance is reduced (FIG. 1A). This correlates to reduced viability and is shown in FIG. 1B relative to starting levels of 100%. Illumination of between 2-5 minutes showed reductions in viability. Increasing TPCS₂ₐ from 0.1 to 1 μg/ml reduced absorbance and cell viability at lower irradiation times and achieved total loss of viability at 4-5 minutes irradiation. It is evident from these results that activation of TPCS₂ₐ resulted in reduced metabolic activity and viability of the treated cells.

FIG. 2 shows the cell density (determined by cell counting) of the treated cells 48 hours after irradiation (FIG. 2A). The higher dose of TPCS₂ₐ reduced cell density at corresponding irradiation times. Higher irradiation times decreased cell density. FIG. 2B shows the cell density relative to starting cell density, i.e. to show relative cell growth.

FIG. 3 shows cell death of the treated cells after irradiation based on Hoechst 33258 staining at 0.1 μg/ml (FIG. 3A) or 1 μg/ml (FIG. 3B) at various irradiation times. The lower dose of TPCS₂ₐ resulted in some cell death but only at longer irradiation times. The higher dose of TPCS₂ₐ resulted in significant cell death after 2 minutes of irradiation.

These results show that the Hoechst stain crossed the plasma membrane. Hoechst 33258 is hydrophilic and does not readily cross the plasma membrane. As such, Hoechst 33258's ability to enter the cell serves as a measure of plasma membrane pore-formation, since pores must exist in the plasma membrane for Hoechst 33258 to enter the cell. The presence of such pores will allow the efflux of molecules for release of contained entities such as viral vectors.

### EXAMPLE 2: LOCALISATION OF PHOTOSENSITISING AGENT (TPCS_{2A}) TO PLASMA MEMBRANE IN SUSPENSION AND ADHERENT CELLS

### Materials and Methods

Jurkat cells (suspension cancer cells of T-cell origin) were incubated with 1 μg/mL fimaporfin (TPCS₂ₐ, tetraphenyl chlorin disulfonate) for 10 minutes in complete RPMI 1640 and HEK293 cells (adherent embryonic kidney cells) were incubated with 5 μg/mL TPCS₂ₐ for 10 minutes in DMEM medium. Cells were subsequently washed in PBS/1% FBS prior to imaging to remove unbound TPCS₂ₐ. The cells were subsequently imaged by light (Nomarski) and fluorescence microscopy to determine the cellular localisation of TPCS₂ₐ.

For imaging, cells were seeded on poly-D-lysine coated cover slips in 24 well plates 1 day prior to treatments. Cover slips were coated with poly-D-lysine for enhanced cell adherence. The coating procedure was performed according to manufacturer's protocol (Thermo Fisher Scientific). Light microscopy (Nomarski) and fluorescence microscopy was performed using Zeiss Imager.Z1. Images were processed using AxioVision. Samples were washed three times in PBS/1% FBS prior to image acquisition in order to remove unbound photosensitiser and thereby visualise cell-bound photosensitiser.

### Results

The localisation of TPCS₂ₐ after incubation with the cells is shown in FIG. 4. The fluorescence images show that TPCS₂ₐ localised to the plasma membrane of both Jurkat and HEK293 cells (see white arrows). It is evident from these results that TPCS₂ₐ localises to the plasma membrane of two highly different cell types (Jurkat and HEK293) after a short incubation period and localises to the same location in both adherent and suspension cells. The incorporation of the photosensitising agent into the plasma membrane allows it to permeabilise the plasma membrane in a cell type-independent manner when activated by irradiation (as shown in the other examples).

### EXAMPLE 3: LEAKAGE OF CYTOSOLIC PROTEIN LACTATE DEHYDROGENASE (LDH) AFTER PHOTOCHEMICAL TREATMENT

Permeabilization of the plasma membrane was assessed using a lactate dehydrogenase (LDH) assay. Such assays are a commonly employed method utilised to indirectly measure cell lysis as LDH is a cytoplasmic enzyme which is released from lysed cells.

### Material and Methods

HEK293 cells were incubated with either 0.5 μg/mL TPCS₂ₐ or 0.5% Tween 20 in serum-free DMEM. Alternatively, HEK293 cells were incubated with 50 μg/mL TPCS₂ₐ in 10% serum supplemented DMEM for 10 minutes. The cells were subsequently irradiated with blue light for 5 minutes or received no irradiation. Blue light irradiation/illumination was performed using LumiSource according to the manufacturer's protocol (PCI Biotech). The negative control (Neg. Ctrl) was either serum-free DMEM or 10% Fetal Bovine Serum (FBS) supplemented DMEM containing the TPCS₂ₐ solvent to control for the effect of solvents on LDH leakage. Background absorbance values of the LDH assay was assessed using DMEM with increasing concentrations of FBS at 1%, 5% and 10%.

LDH release was performed as a measure of cell lysis and release of cytosolic material using CyQUANT LDH Cytotoxicity Assay (Thermo Fisher Scientific) according to manufacturer's protocol. Cells were seeded in 48 well plates one day prior to treatments in 400 μL. The following day, treatments were given for 10 minutes at 37° C. 5% CO₂, prior to 5 minutes illumination. Plates were subsequently incubated for 2 hours in an incubator (37° C. 5% CO₂), then centrifuged for 5 minutes at 400×g, after which 50 μL supernatant from each sample was transferred to a 96 well plate for absorbance measurements according to kit instructions. 490 nm absorbance reflects LDH and 680 nm reflects absorbance background from the instrument. 490 nm absorbance minus 680 nm absorbance is directly proportional to the amount of LDH released into the medium.

### Results

FIG. 5 shows LDH release from HEK293 cells treated with 0.5 μg/mL TPCS₂ₐ or Tween 20 (FIG. 5A) or 50 μg/mL TPCS₂ₐ (FIG. 5B). In the absence of irradiation, HEK293 cells in both serum-free and 10% FBS DMEM treated with TPCS₂ₐ exhibited comparable levels of LDH release to HEK293 cells treated with a negative control. In the presence of irradiation, LDH release was increased significantly in both serum-free and serum-supplemented medium. In contrast, HEK293 cells treated with Tween 20, a commonly used detergent which causes cell lysis, showed elevated levels of LDH release both in the absence and presence of irradiation.

It is evident from these results that treatment with TPCS₂ₐ allows for light-dependent LDH release, and therefore cell permeabilisation (and potentially lysis), in both serum-free DMEM and 10% FBS DMEM. The results of FIG. 5C further show that elevated concentrations of FBS within the DMEM serum results in increased absorbance values in the LDH assay. This therefore demonstrates that the overall higher absorbance values seen in FIG. 5B compared with FIG. 5A can be attributed to the presence of 10% FBS in FIG. 5B.

### EXAMPLE 4: ASSESSMENT OF MORPHOLOGY AFTER PHOTOCHEMICAL TREATMENT

### Materials and Methods

HEK293 cells were incubated with 5 μg/mL TPCS₂ₐ complete DMEM for 10 minutes, followed by irradiation with blue light for 5 minutes. Blue light irradiation/illumination was performed using LumiSource according to the manufacturer's protocol (PCI Biotech). Cells were imaged by light (Nomarski) and fluorescence microscopy prior to illumination and 2 hours after illumination. Imaging was performed as described in Example 2.

### Results

The impact of photochemical lysis in HEK293 cells treated with TPCS₂ₐ can be seen in FIG. 6 which demonstrates that in the absence of irradiation, HEK293 cells treated with 5 μg/mL TPCS₂ₐ do not exhibit changes to their cellular morphology. In contrast, HEK293 cells treated with 5 μg/mL TPCS₂ₐ which had been irradiated exhibited a dramatically altered cellular morphology and had shifted to a lytic phenotype, with cellular material being scattered outside the remaining cellular structures (cell corpses).

### EXAMPLE 5: SELECTIVE PERMEABILIZATION OF THE PLASMA MEMBRANE OF HEK293 CELLS BY IRRADIATION AFTER TREATMENT WITH A PHOTOSENSITISING AGENT, TPCS_{2A}

The relative effects of photochemical treatments and detergent lysis on release of cellular components were assessed.

### Materials and Methods

HEK293 cells were incubated with 5 μg/mL TPCS₂ₐ or 0.5% Tween 20 in complete DMEM for 10 minutes followed by irradiation for 5 minutes. Blue light irradiation/illumination was performed using LumiSource according to the manufacturer's protocol (PCI Biotech). A negative control of complete DMEM containing the TPCS₂ₐ solvent was used to control for solvent effects on cell morphology and lysis.

Hoechst 33258 stain was added to samples 2 minutes prior to imaging. Hoechst 33258 staining was performed according to the manufacturer's protocol (Thermo Fisher Scientific). The cells were imaged by light (Nomarski) and fluorescence microscopy prior to irradiation and 2 hours after irradiation. Imaging was performed as described in Example 2. Changes to cellular morphology were analysed visually.

### Results

DNA leakage from cells is a major problem in viral vector manufacturing, specifically the leakage of genomic DNA from producer cells. To understand how TPCS₂ₐ treatment and established lysis methods (Tween 20) impacted DNA leakage, cells lysed by both approaches were studied by microscopy. Hoechst 33258 staining was used to stain free DNA or DNA in cells with plasma membrane pores. The results in FIG. 7 show that while DNA stays within the boundaries of the cell corpse following TPCS₂ₐ treatment and irradiation, detergent lysis with Tween 20 causes DNA leakage from cells after 10 minutes, and after 2 hours DNA is free in solution.

It is evident from these results that TPCS₂ₐ treatment may be employed to selectively lyse cells without resulting in DNA leakage and contamination, in contrast to detergent lysis.

### EXAMPLE 6: RELEASE OF VIRAL VECTORS FROM PRODUCER CELLS BY IRRADIATION AFTER TREATMENT WITH A PHOTOSENSITISING AGENT, TPCS_{2A}

Photochemical lysis was used to release AAV2 viral vectors from adherent HEK293T cells in which they were produced.

### Materials and Methods

75-80% confluent HEK293T cells were triple transfected with Adeno-associated virus serotype 2 (AAV)-encoding plasmids (pHelper, AAV2 RepCap, and pscAAV-GFP plasmids) using polyethylenimine (PEI) in 12 well plates, resulting in AAV2 production. For each transfection, plasmids and PEI were added to complete DMEM to a total volume of 640 μL, followed by vortexing for 10 seconds and 15 minutes incubation at room temperature. Medium was removed from the HEK293T cells in 12 well plates, and replaced by the DMEM-plasmid-PEI mixture. Cells were incubated for three days at 37° C. and 5% CO₂. Three days after transfection, cells were subjected to treatments (as described below) for 10 minutes in complete DMEM.

Treatments were:
a) Untransfected cells, no further treatment.
b) Transfected cells. Received photosensitiser solvent but no photosensitiser (to control for solvent effect on cell lysis).
c) Transfected cells. Incubated with 5 μg/mL TPCS₂ₐ (Fimaporfin) for 10 minutes.

Samples from treatment b) or c) were previously (on the day of seeding) divided into two 12-well plates and either subjected to blue light illumination for 5 minutes or no light illumination. Blue light irradiation/illumination was performed using LumiSource according to the manufacturer's protocol (PCI Biotech).

Supernatants were harvested 2 hours later and cell debris (if any) was eliminated by centrifugation. DNase-resistant viral genome (vg) from all samples was quantified by digital droplet PCR amplification (Bio-Rad QX600) of DNase-resistant (i.e. viral capsid-encapsulated) DNA using primers targeting inverted terminal repeats. Viral vector yield was expressed as “vg/mL” (AAV vector genomes per millilitre).

The ITR primers' sequences are as set out below:

ITR forward:
(SEQ ID NO: 1)
CGGCCTCAGTGAGCGA
ITR reverse:
(SEQ ID NO: 2)
GGAACCCCTAGTGATGGAGTT

### Results

The results are shown in FIG. 9. “No Trf” shows supernatants from untransfected cells. “Neg. Ctrl” shows supernatant from transfected cells that received photosensitiser solvent but no photosensitiser. “Fimaporfin” shows supernatant from transfected cells that were incubated with TPCS₂ₐ for 10 minutes. No light shows samples there was not illuminated and with light shows samples that were illuminated.

The figure shows that photochemical lysis releases non-enveloped viral vectors from producer cells (here, HEK293T, the most common cell type). Whilst this experiment is concerned with AAV2, this virus is representative of other non-enveloped vectors (e.g. other AAV serotypes and adenovirus (AV)) to which the method may be applied.

### EXAMPLE 7: PHOTOCHEMICAL LYSIS OF HEK293T SUSPENSION CELLS

The impact of photochemical treatments and detergent lysis on HEK293T suspension cells was studied by microscopy.

### Materials and Methods

HEK293T suspension cells were treated with 5 μg/mL TPCS₂ₐ (fimaporfin) or fimaporfin solvent without photosensitiser (“Neg. Ctrl”) in complete DMEM for 10 minutes, followed by blue light illumination for 5 minutes (as described in Example 6). Hoechst 33258 was added to samples 2 minutes prior to imaging. Hoechst 33258 DNA stain is not readily cell penetrable, and therefore stains free DNA or DNA in cells with plasma membrane pores.

Cells were washed in PBS/1% FBS prior to imaging to remove unbound fimaporfin. Cells were imaged by light (Nomarski) microscopy and fimaporfin and Hoechst fluorescence by fluorescence microscopy after 10 minutes (i.e. prior to illumination) and 2 hours after illumination. Imaging was conducted as set out in Example 2.

### Results

The results are shown in FIG. 10. Arrows indicate the plasma membrane localisation of fimaporfin (prior to illumination, i.e. after 10 minutes). Asterisks indicate where the plasma membrane has been disrupted and the corresponding absence of fimaporfin staining. Plus signs indicate Hoechst positive DNA and the corresponding localisation of DNA in lysed cell corpses.

These results are in line with those observed in FIG. 4 (fimaporfin plasma membrane localisation in Jurkat and adherent HEK293), 6 (plasma membrane disruption of adherent HEK293), and 7 (retention of DNA following photochemical lysis, adherent HEK293) but with HEK293T suspension cells, the most commonly used cell type in viral vector manufacturing performed in suspension culture. The key results are: 1) fimaporfin localises to the plasma membrane of HEK293T suspension cells, 2) photochemical lysis (fimaporfin+light) opens up the plasma membrane of HEK293T suspension cells, and 3) DNA is retained within the boundaries of the lysed cells (“cell corpses”) after photochemical lysis.

### EXAMPLE 8: SELECTIVE PERMEABILIZATION OF THE PLASMA MEMBRANE OF HEK293T CELLS BY IRRADIATION AFTER TREATMENT WITH A PHOTOSENSITISING AGENT, TPCS_{2A}, STUDIED BY DIGITAL DROPLET PCR

The relative effects of photochemical treatments and detergent lysis on release of cellular components were assessed by digital droplet PCR (ddPCR).

### Materials and Methods

75-80% confluent HEK293T cells were incubated with 5 μg/mL TPCS₂ₐ or 0.5% Tween 20 in complete DMEM for 10 minutes followed by irradiation for 5 minutes (where indicated). Blue light irradiation/illumination was performed using LumiSource according to the manufacturer's protocol (PCI Biotech). A negative control of complete DMEM containing the TPCS₂ₐ solvent was used to control for solvent effects on cell lysis.

Following irradiation, the cells were incubated for 2 hours at 37° C. and 5% CO₂, after which supernatants were collected. Cell debris (if any) was eliminated by centrifugation. Genomic DNA from all samples was quantified by ddPCR (Bio-Rad QX600) using primers targeting human albumin DNA.

The primers' sequences are as set out below:

Albumin forward:
(SEQ ID NO: 3)
TGAAACATACGTTCCCAAAGAGTTT
Albumin reverse:
(SEQ ID NO: 4)
CTCTCCTTCTCAGAAAGTGTGCATAT

Albumin DNA values were normalised to the negative control.

### Results

The results in FIG. 11 show that a photochemical lysis condition known to lyse cells and release viral vectors (Example 6) does not cause the leakage of genomic DNA from HEK293T cells. In contrast, Tween 20, an established lysis method, caused an approximately 6-fold increase in genomic DNA.

The results reported in FIG. 7 provide qualitative evidence that photochemical lysis, in contrast to Tween 20 lysis, does not cause genomic leakage. The present results quantitatively confirm that photochemical lysis does not open up the nucleus of producer cells, preventing a strong leakage of genomic DNA unlike with Tween 20 and detergent lysis. Therefore, TPCS₂ₐ treatment may be employed to selectively lyse cells without resulting in DNA leakage and contamination, in contrast to detergent lysis.

### EXAMPLE 9: ASSESSMENT OF PHOTOCHEMICAL LYSIS WITH ALTERNATIVE PHOTOSENSITISERS

The effects of five photochemical treatments on morphology and release of cellular components were assessed.

### Materials and Methods

HEK293T cells were incubated with 5 μg/mL verteporfin (a benzoporphyrin), 0.03 μg/mL temoporfin (a chlorin), 3 μg/mL chlorin E6 (a chlorin), 30 μg/mL protoporphyrin IX (a porphyrin), or 10 μg/mL AIPcS₂ₐ (a phthalocyanine) in complete DMEM for 10 minutes followed by irradiation for 5 minutes. Blue light irradiation/illumination was performed using LumiSource according to the manufacturer's protocol (PCI Biotech).

Hoechst 33258 stain was added to samples 2 minutes prior to imaging to stain free DNA or DNA in cells with plasma membrane pores. Hoechst 33258 staining was performed according to the manufacturer's protocol (Thermo Fisher Scientific). The cells were imaged by light (Nomarski), and Hoechst and photosensitiser fluorescence imaged by fluorescence microscopy after 10 minutes (i.e. prior to irradiation) and 2 hours after irradiation. Imaging was performed as described in Example 2.

### Results

The impact of photochemical lysis in HEK293T cells treated with five alternative photosensitisers reproduces what has already been shown using TPCS₂ₐ in Examples 4 and 5. FIG. 12 shows that a short incubation with each of the tested photosensitisers can lyse cells in a light-dependent manner (demonstrated by morphological changes in the Nomarski images before and after illumination) and that photochemical lysis prevents DNA leakage from the lysed cells (demonstrated by the round, Hoechst 33258-positive shapes that appear after illumination which are nuclei, or nuclei-like structures).

Together with the results in Examples 4 and 5, these results illustrate that numerous classes of photosensitisers (benzoporhyrin, porphyrin, phthalocyanine, chlorin) are capable of producing photochemical lysis, demonstrating that photochemical lysis to achieve cellular release of viral vectors is a general principle and is not specific to fimaporfin.

1. A method of releasing a viral vector from a cell in which said viral vector has been produced, comprising:
a) contacting a cell in which said viral vector is present with a photosensitising agent,
b) irradiating said cell with light of a wavelength effective to activate said photosensitising agent, wherein said irradiation is conducted at a dose of light and for a time sufficient to disrupt the plasma membrane of said cell, thereby releasing said viral vector, and
c) optionally collecting and/or purifying said released viral vector.

2. The method of claim 1, wherein said cell is a mammalian cell, preferably a human cell.

3. The method of claim 1, wherein said cell is selected from a HEK293 cell, a Vero cell, a sf9 cell and a PER.C6 cell.

4. The method of any one of claims 1 to 3, wherein the viral vector is a virus that lacks an envelope, preferably an adenovirus or an adeno-associated virus.

5. The method of any one of claims 1 to 4, wherein the photosensitising agent is an amphiphilic or hydrophobic photosensitising agent, preferably TPCS₂ₐ or TPPS₂ₐ.

6. The method of any one of claims 1 to 5, wherein the light has a wavelength of 400-700 nm (visible) or 400-475 nm (blue light).

7. The method of any one of claims 1 to 6, wherein said contacting step a) is performed for 0.5-120 minutes, preferably 2-30 minutes.

8. The method of any one of claims 1 to 7, wherein the irradiation in step b) is performed for 0.5-120 minutes.

9. The method of any one of claims 1 to 8, wherein a lysing agent is added to said cell in step a), b) and/or c).

10. The method of any one of claims 1 to 9, wherein the cell is in an aqueous medium during steps a) and b).

11. The method of any one of claims 1 to 10, wherein the plasma membrane is disrupted by the generation of pores in said membrane.

12. The method of any one of claims 1 to 11, wherein at least 30% of the genomic DNA in said cell prior to illumination remains in said cell after release of said viral vector.

13. The method of any one of claims 1 to 12, wherein said collection is by removal of cell debris.

14. The method of claim 13, wherein the cell is in an aqueous medium during steps a) and b) and collection is performed by separation of the aqueous medium from cell debris which is not suspended in said medium, preferably by centrifugation.

15. The method of any one of claims 1 to 14, wherein said viral vector is subject to purification, preferably using at least one of the following methods selected from centrifugation, sonication, freeze-thawing, enzyme digestion and liquid chromatography, preferably to a purity of at least 50% (w/w, dry weight).

16. The method of any one of claims 1 to 15, wherein prior to said contacting step a) a step in which said viral vector is produced in said cell is performed.

17. The method of claim 16, wherein said viral vector is produced by culturing said cell to allow said cell to produce said viral vector and optionally the culture supernatant is removed before step a).

18. The method of claim 17, wherein said cell produces said viral vector after infection of said cell with one or more of said viral vectors and/or transfection with one or more polynucleotides which allow the production of said viral vector in said cell.

19. The method of any one of claims 16 to 18, wherein prior to said step in which said viral vector is produced a step is performed in which said cell is infected with one or more of said viral vectors or transfected with one or more polynucleotides and/or viral vectors which allow the production of said viral vector in said cell.

20. A cell harvesting kit or apparatus for releasing a viral vector from a cell comprising:
a) a photosensitising agent; and
b) a light source to irradiate said cell.

21. The kit or apparatus of claim 20, wherein said kit or apparatus additionally comprises a container in which said cell may be contained.

22. The kit or apparatus of claim 21, wherein said container is suitable for cell culture or purification of said cell.

23. The kit or apparatus of claim 21 or 22, wherein said container is a bag or a tank and/or said light source is attached to said container.

24. The kit of any one of claims 20 to 23, wherein said kit or apparatus additionally comprises a means to agitate said cells.

25. A preparation of viral vectors obtainable by a method as defined in any one of claims 1 to 19.
